(19) United States
(12) Patent Application Publication
(10) Pub. No.: US 2025/0128042 A1
(43) Pub. Date: Apr. 24, 2025

(54) HEMOCOMPATIBLE OPTICAL SENSOR FOR A MEDICAL DEVICE

(71) Applicant: ABIOMED, Inc., Danvers, MA (US)

(72) Inventor: Dustin Seth West, Danvers, MA (US)

(73) Assignee: ABIOMED, Inc., Danvers, MA (US)

(21) Appl. No.: 18/919,861

(22) Filed: Oct. 18, 2024

Related U.S. Application Data
(60) Provisional application No. 63/591,355, filed on Oct. 18, 2023.

(51) Int. Cl. A61M 60/00

(52) U.S. Cl. CPC A61M 60/00; A61M 2205/3306

(57) ABSTRACT

An optical assembly for placement on a medical device. The optical assembly has a diaphragm suspended on and over an optical cavity. The optical cavity has a sensor dot formed thereon. The diaphragm is covered with a biocompatible platinum silicide layer.

### CROSS-REFERENCE TO RELATED APPLICATIONS

This application claims priority to and the benefit of U.S. Provisional Application No. 63/591,355 which was filed on Oct. 18, 2023 and is incorporated by reference herein in its entirety.

### TECHNICAL FIELD

Described herein is a medical device with an optical sensor affixed thereto.

### BACKGROUND

An intravascular blood pump assembly, such as an assembly with an intracardiac blood pump, may be introduced into the heart to deliver blood from the heart into an artery. Intravascular blood pumps can be introduced percutaneously during a cardiac procedure through the vascular system, such as by a catheterization procedure. Some blood pumps are designed to support the left side of the heart, where they pull blood from the left ventricle of the heart and expel the blood through a cannula into the aorta. Some blood pumps that support the left side of the heart are introduced by a catheterization procedure through the femoral artery, into the ascending aorta, across the aortic valve, and into the left ventricle.

### BRIEF SUMMARY

Described herein is a medical device having an optical sensor attached thereto. The optical sensor is formed on a glass substrate. The optical sensor has a biocompatible platinum silicide layer formed over a diaphragm formed over and supported by a glass substrate with an optically reflective cavity therein. Multiple sensors are formed on a single substrate and singulated into individual devices. One or more individual devices are then placed on the medical device.

Described herein is an optical sensor assembly for use in a blood pump assembly comprising a visor having an inner surface and an outer surface, a support jacket in contact with the inner surface of the visor, an optical sensor disposed within the support jacket, and a protective layer being deposited over and covering the diaphragm of the optical sensor assembly.

The optical sensor comprises an optical sensor inner surface and a diaphragm disposed over a cavity in a substrate. The diaphragm has an exposed surface and exposed sides. The protective layer being deposited over and covering the exposed surface and exposed sides of the diaphragm. The protective layer is one of a platinum-containing layer, a platinum silicide layer, or parylene.

Also described herein is a method of fabricating an optical sensor comprising forming a silicon layer over a glass substrate via masking, defining a cavity between the silicon layer and the glass substrate, and forming a protective layer over the silicon layer and over an uncovered edge portion of the silicon layer.

### BRIEF DESCRIPTION OF DRAWINGS

A brief description of the drawings follows.

FIG. 1 is a cross section of a prior art sensor device.

FIG. 2 illustrates a substrate on which multiple sensors are formed.

FIG. 3A-3C illustrates one method for forming a prior art optical sensor.

FIG. 4A-4E illustrates one aspect for fabricating the optical sensor described herein.

FIG. 5A illustrates a pump assembly with the optical sensor described herein.

FIG. 5B illustrates an interface between the pump assembly and the optical sensor described herein.

FIG. 6 illustrates an optical sensor assembly for use in a blood pump assembly.

### DETAILED DESCRIPTION

Embodiments of the present disclosure are described in detail with reference to the drawing figures wherein like reference numerals identify similar or identical elements. It is to be understood that the disclosed embodiments are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

To provide an overall understanding of the systems, method, and devices disclosed herein, certain illustrative implementations will be described. Although the implementations and features described herein are specifically described for use in connection with a blood pump assembly, it will be understood that the teaching may be adapted and applied to other pumps and other types of medical devices.

Blood pumps may be inserted into the left ventricle to support the left side of the heart. Some systems are designed to support the right side of the heart, where the blood pump is introduced through a vein and into the right side of the heart through the venous system (i.e., the vena cava). Such blood pump systems may also be surgically implanted or inserted through the subclavian and/or carotid arteries. During the insertion of a blood pump assembly into a patient through a blood vessel, it may be difficult to advance the blood pump through the tortuous paths and/or calcified anatomy of the patient.

Complications involving the introduction of the pump due to these tortuous paths may, in some cases, cause damage to the blood pump assembly, or to the patient. A damaged blood pump may need to be removed or replaced, or it may no longer be accurate, or operational. For example, damage to pump sensors may prevent accurate pump introduction or operation.

The blood pump's sensors (e.g., an optical sensor) can be particularly vulnerable to damage during insertion or operation of the pump. For example, the forces exerted on such a sensor deployed with a blood pump within a patient can cause the sensor to crack. Additionally, prolonged exposure to the complex biological environment may lead to several mechanisms that degrade sensor performance such as biological deposits forming on sensor elements, erosion of sensing elements, or opto-mechanical changes of sensing elements. Damage to the optical sensor can prevent the sensor from conveying to the practitioner the important signals picked up by the sensor.

One approach protects the optical sensor by applying a layer of silicone gel to the surface of the sensor. Additional layers of silicone gel may provide increased protection. However, silicone gel may be unstable and/or migrate because of an applied load. Instability of the gel can degrade sensor performance. Migration of the silicone gel will degrade sensor performance and may impair the adhesion of the optical sensor to the pump housing.

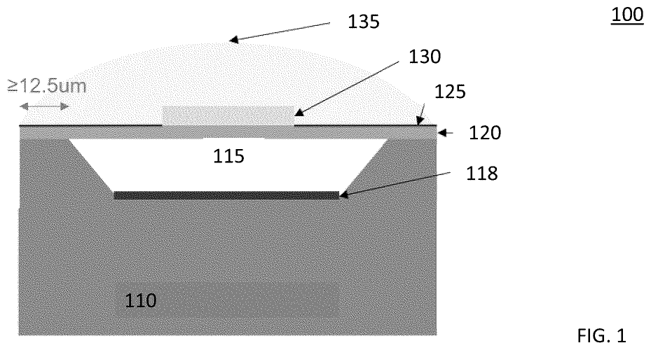

An example of such a sensor is illustrated in FIG. 1. The sensor is formed on a glass substrate 110. The substrate 110 has a cavity 115 formed therein. A diaphragm 120 is formed over the cavity 115 and is supported by the surface of the substrate 100. As will be appreciated, the silicon diaphragm 120 may be formed of any suitable material. A mirror 118 is deposited on the bottom of the cavity 115 and acts as a partial mirror for light that is delivered to device 100. Consequently, light directed to the diaphragm 120 is either transmitted through or reflected by the diaphragm 120. In some embodiments, a protective layer 125 is formed over the diaphragm 120 and the dot 130 and the glass substrate 110. In some embodiments, a protective layer 125 is formed over the diaphragm 120 and the dot 130. In some embodiments, the protective layer 125 is formed over the diaphragm 120 except for the region over which the dot 130 is formed. In some embodiments, a dot 130 may not be included. As will be appreciated, the protective layer 125 may be any suitable material. The protective layer 125 may be an inert thin film material. For example, the protective layer 125 may be parylene. In another example, the protective layer 125 may be titanium. The protective layer 125 may be platinum. The protective layer 125 may combine (e.g., alloy) with the silicon membrane. The dot 130 may be silicon dioxide. In some embodiments, a biocompatibility layer 135 is formed over the surface of the device 100. In some embodiments, the biocompatibility layer may be silicone material. For example, the biocompatibility layer may be a silicone gel material. However, as will be appreciated, the biocompatibility layer may be of any suitable material. In some embodiments, the protective layer 125 and the biocompatibility layer 135 may be the same layer.

The diaphragm is highly susceptible to biochemical attack in blood and thereby degrading the optical sensor. Additionally, the sides of the diaphragm may not be covered by the protective layer and/or the biocompatibility layer due to the geometry of the diaphragm. The diaphragm may extend to the edges of the glass substrate and thus it may be difficult for the protective layer and/or biocompatibility layer to cover the edges of the diaphragm. Furthermore, when the diaphragm is extended to the edges of the glass substrate, it may be prohibitively difficult to protect the diaphragm with an applied liquid due to the liquid surface tension of the applied protective layer and/or biocompatibility layer causing it to pull away from the edges before curing. The diaphragm edges may be thus left exposed and may be susceptible to damage and/or failure.

Accordingly, it is recognized herein that it would be desirable to have an improved optical sensor assembly that provides one or more of the advantages of protecting the sensor during pump insertion, mitigating adhesion challenges of the sensor to the pump housing, preventing corrosion during long durations of use of the optical sensor assembly components when the blood pump assembly is deployed within the patient. Furthermore, the benefit of modifying the geometry of the diaphragm to allow for the edges of the diaphragm to be protected. Furthermore, the benefit of sensor singulation (e.g. dicing) with some distance between the side(s) of the diaphragm and the singulation feature(s) is recognized herein.

Thus, the systems, methods, and devices described herein provide a blood pump assembly including a sensor and a protective layer that protects the sensor from physical and/or chemical damage. The sensor may include a diaphragm, which may be fragile. The protective layer covers the diaphragm and enables the blood pump assembly and sensor to resist biochemical degradation from the blood of the patient and/or increases durability and reliability of the optical sensor. For example, the protective layer may prevent the diaphragm from being dissolved by chemical reactions with a patient's blood without significantly influencing or interfering with accurate detection of pressure. The systems, methods and devices described herein also provide a diaphragm which is covered on all exterior surfaces with a protective layer to further increase reliability and durability.

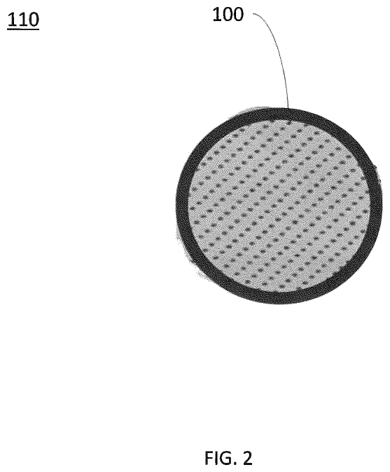

As described, the optical sensors are devices formed as an array on a glass substrate 110 as illustrated in FIG. 2. The optical sensor devices 100 are visible as “dots” on the substrate 110.

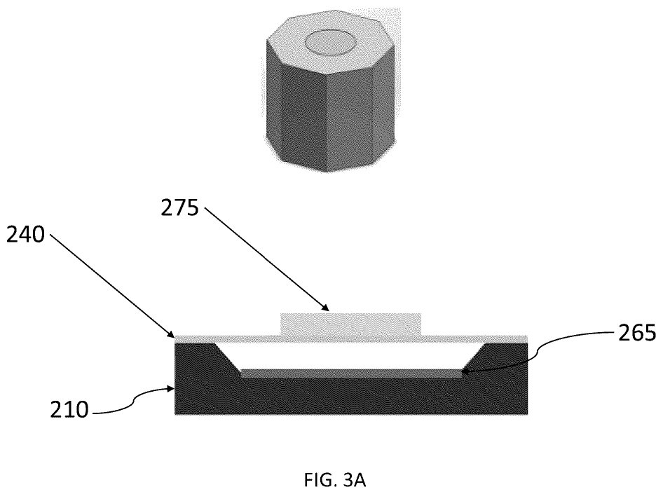

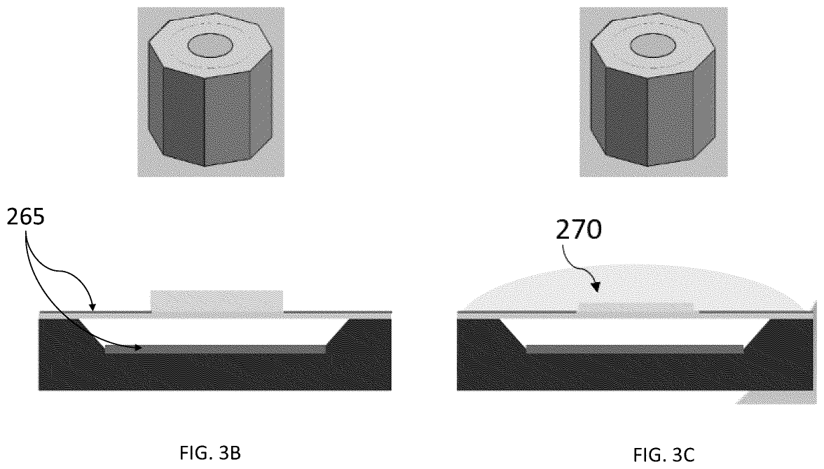

The method for fabricating the sensor as illustrated in FIG. 1 is illustrated in FIGS. 3A-3C. A cavity may be formed between the silicon layer 240 and the glass substrate 210. As will be appreciated, the silicon layer 240 may be formed using any suitable means. For example, the silicon layer 240 may be formed via anodic bonding and may include etching operations to refine the geometry. In some embodiments, a protective layer 265 may cover the silicon layer 240 and may extend beyond to cover the glass substrate 210 in whole or in part. As will be appreciated, layer 265 may be made of any suitable material. For example, layer 265 may be made of a ceramic material. As seen in FIG. 3A, a dot 275 is defined on top of the silicon layer 240. As will be appreciated, the dot 275 may be formed using any suitable means. For example, the dot may be formed via masking. A protective layer 265 is formed over the top of the silicon layer 240 as seen in FIG. 3B. As seen in FIG. 3C, a protective layer 270 is formed.

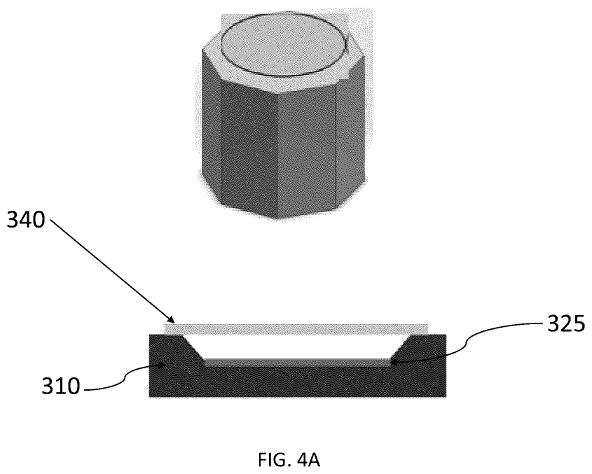

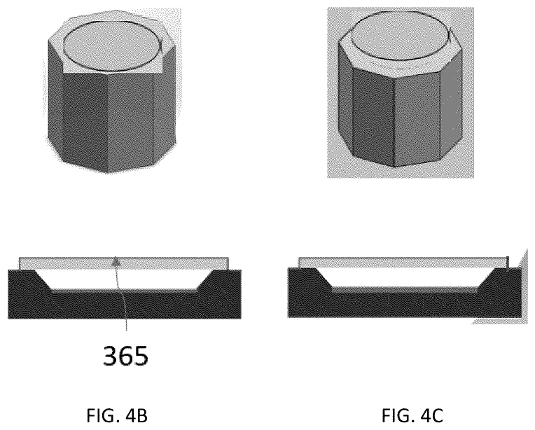

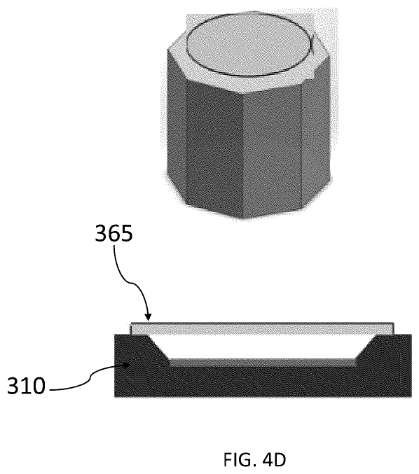

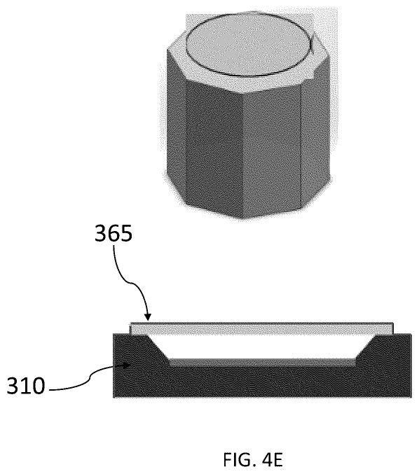

A method for fabricating the sensor described herein is illustrated in FIGS. 4A-4E. In FIG. 4A, a silicon layer 340 may formed. A cavity may be formed between the silicon layer 340 and the glass substrate 310. The silicon layer 340 may be formed over the glass substrate 310. As will be appreciated, the silicon layer 340 may be formed using any suitable means. For example, the silicon layer 340 may be formed via masking. In some embodiments, a layer 325 may also be included on the glass substrate in the cavity to reflect light. As will be appreciated, layer 325 may be made of any suitable material. For example, the layer 325 may be a ceramic material. As illustrated in FIG. 4B, a protective layer of platinum silicide 365 is formed over the structure in FIG. 4A. Due to the biocompatibility of the platinum layer, there may be no requirement for a silicone protective layer. In some embodiments, the protective layer may be platinum. As will be appreciated, platinum silicide can be formed using any suitable means. For example, in some embodiments, platinum silicide may be formed by depositing a layer of platinum over a layer of silicon and increasing the temperature. In some embodiments, the protective layer may be parylene. As will be appreciated, any type of parylene may be used. In some embodiments, the sensor shown in FIGS. 4A-4C may include a dot.

The diaphragm also has a geometry that permits both the top and edge surfaces to be covered with the protective layer. In some embodiments, the diaphragm may not extend to the edge of the glass substrate 310, as shown in FIG. 4A-4C. In such embodiments, the edges of the diaphragm may be exposed during the step of applying the protective layer and thus the edges of the diaphragm may be covered with the protective layer, as seen in FIG. 4C. In some embodiments, the exposed edges of the top of the glass substrate may not be covered with platinum, as seen in FIG. 4D. In other embodiments, the vertical sides of the glass substrate may also be covered with a platinum layer, as seen in FIG. 4E. As will be appreciated, the diaphragm may be of any suitable cross-sectional geometry. For example, the diaphragm may be of a circular cross-sectional geometry, as shown in FIG. 4A-4C. In another embodiment, the diaphragm may have an octagonal cross-sectional geometry.

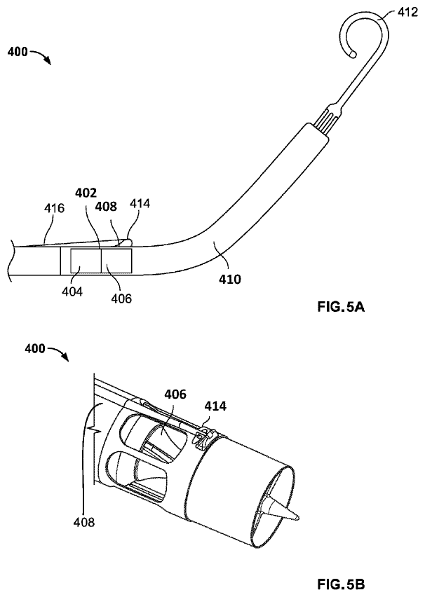

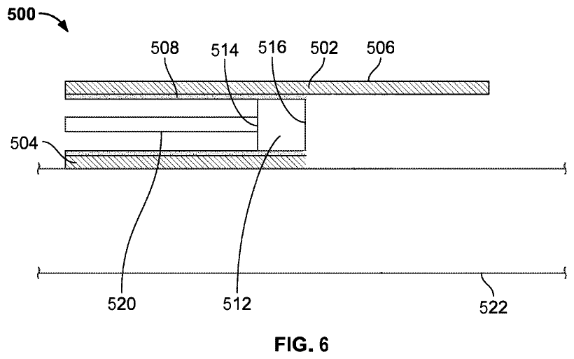

FIG. 5A shows an illustrative blood pump assembly 400 having a pump 402, a motor 404, a rotor 406, pump housing 408, a cannula 410, an atraumatic extension 412, and an optical sensor assembly 414. The optical sensor assembly 414, as described further in relation to FIG. 6 below, comprises a visor, a support jacket, an optical sensor, at least one layer of a protective layer, and an optical fiber 416. The pump 402 comprises the motor 404 and rotor 406. The rotor 406 has at least one blade for conveying fluid through pump 402. The pump housing 408 is configured to surround the at least one blade of rotor 406. The cannula 410 extends from the pump housing 408 in a distal direction. In some embodiments, the cannula 410 may be expandable. In some embodiments, the atraumatic extension 412 extends from the cannula 410 in a distal direction. In other embodiments, the blood pump assembly 400 may not include an atraumatic extension 412. In certain implementations, the atraumatic extension 412 is a pigtail. The optical sensor assembly 414 is configured to bind to the pump housing 408 by the visor of the optical sensor assembly 414. In some embodiments, there may be more than one optical sensor assembly 414. As will be appreciated, the optical sensor assembly 414 may be positioned in any suitable location on the blood pump assembly 400. For example, as shown in FIG. 5B, the optical sensor assembly 414 may be positioned distal to the pump housing 408.

FIG. 5B shows an illustrative interface between the pump housing 408 and the optical sensor assembly 414. The means of adhesion between the optical sensor assembly 414 and the pump housing 408 may be selected in order to tailor the strength of the bond between the optical sensor 414 and pump housing 408. The bond strength is advantageously selected based on the shear forces that will be exerted on the pump by the blood during operation and insertion of the pump. For example, a weak bond between the optical sensor assembly 414 and pump housing 408 may cause the two components to separate when subject to shear forces that exceed the bond strength. In some implementations, the visor of the optical sensor assembly 414 may be bound to the pump housing 408 by a glue. In certain implementations, the glue is a 2-part epoxy. In other implementations, the glue is a UV light-bonded glue. In further implementations, the visor is fused to the pump housing 408. In some implementations, the epoxy used to bond the visor to the pump housing is selected based on the tackiness of the epoxy, with larger values of tackiness corresponding to stronger bonds between the visor and pump housing 408. The tackiness of a substance can be measured by prodding the substance with a probe and determining the energy required to break the bond formed be between the substance and the probe. Such measurements yield tack energies of a substance, with larger tack energies corresponding to tackier substances that form bonds that require more energy to break. In certain implementations, the tack energy of the epoxy is between about 2 J/cm² and about 10 J/cm². In other implementations, the tack energy of the epoxy is between about 4 J/cm² and about 8 J/cm². In further implementations, the tack energy of the epoxy is about 6 J/cm². Additionally, a larger amount of a given epoxy used to bind the visor to pump housing 408 corresponds to a stronger bond between the visor and pump housing 408. The optical sensor assembly 414 may be further welded to pump housing 408. Additionally, the visor may be alternately glued to pump housing 408 and welded to the pump housing 408 in different regions along the area of the visor. At least one advantage of the configuration of the visor to be glued or fused to pump housing 408 is that both methods of adhesion can be employed in order to provide the strongest bond between the visor and pump housing 408.

FIG. 6 shows an illustrative optical sensor assembly 500 for use in a blood pump assembly (e.g., blood pump assembly 400 of FIG. 5A). In some embodiments, the optical sensor assembly 500 may include a visor 502 having a visor inner surface 504 and a visor outer surface 506, an optical sensor 512 having an optical sensor inner surface 514 and an optical sensor diaphragm 516, an optical fiber 520, and a pump housing 522. Support jacket 508 surrounds the visor outer surface 506 and visor inner surface 504. Support jacket 508 is removed during manufacturing, leaving only the visor outer surface 506 and visor inner surface 504. In some implementations, the support jacket 508 may comprise a polymer tube. The optical sensor 512 may be disposed within the visor 502. In some implementations, the optical sensor inner surface 514 may be connected to the optical fiber 520. In other implementations, the optical sensor inner surface 514 may be connected to the visor inner surface 504. In certain implementations, the optical diaphragm 516 may be configured to receive a protective layer, as will be described in greater detail below. In some embodiments, the optical sensor assembly 500 may not include a visor 502.

The diaphragm 516 of the sensor 512 is configured to deflect in response to changes in blood parameters, for example, changes in pressure, flow rate, fluid composition, and/or viscosity. The diaphragm 516 is preferably thin. In some embodiments, the diaphragm 516 is less than two microns thick. In some embodiments, the diaphragm 516 may be composed of a material such as silicon. As will be appreciated, the diaphragm 516 may be any suitable material. Deflections of the diaphragm 516 are used to measure changes in blood parameters (for example, blood pressure) at the blood pump assembly 400. Due to the bend radius constraints of the transmission fiber, the diaphragm 516 points forward towards the distal end of the blood pump assembly 400. Deflections of the diaphragm 516 are sensed by a sensor head of the sensor 512 and transmitted to the optical fiber 520.

As discussed herein, the blood pump assembly 400 may be introduced percutaneously during a cardiac procedure through the vascular system. For example, the blood pump assembly 400 can be inserted by a catheterization procedure through the femoral artery, into the ascending aorta, across the valve, and into the left ventricle such that the blood pump assembly 400 can provide support to the left side of the heart. As noted, introducing the blood pump assembly 400 through an introducer unit into the vascular system may include traversing torturous directional changes and a calcified anatomy in the vascular system. The sensor, and in particular the diaphragm 516, may be composed of sensitive or brittle components that may be easily damaged by the torturous and calcified anatomy of the vascular system. The visor and protective layer and/or biocompatibility layer permit the sensor to traverse the torturous and calcified anatomy of the vascular system and remain operable. For example, the visor and protective layer and/or biocompatibility layer may protect the diaphragm 516 by preventing soft obstructions, such as valve leaves on a blood pump introducer, from contacting and damaging the diaphragm. In some embodiments, the protective layers and/or biocompatibility layers on the diaphragm may increase the reliability of the sensor by protecting the diaphragm from corrosion from the patient's blood.

As shown in FIG. 4F, the sensor head includes the cavity. The optical fiber 520 ends in the proximal portion of the sensor head to which it is coupled with low loss. The cavity in combination with the diaphragm forms a Fabry-Perot resonator. To allow for the resonant measuring principle, both sides of the cavity are manufactured to reflect light. As far as detection of the signal has to be made possible, on one side, preferably the optical fiber side, partial reflection is realized, and preferably on the diaphragm side, full reflection is realized. The jacket is positioned about the optical fiber and may include a glass ring. The optical fiber is coupled to the sensor head by a glue, which may be ultraviolet-curing epoxy. In some embodiments, the visor is configured to extend beyond a distal end of the diaphragm.

The protective layer may include material capable of being deposited onto the diaphragm. In this embodiment, the protective layer is made of platinum to improve hemocompatibility and durability of the optical sensor. As will be appreciated, the protective layer material composition may include any suitable amount of platinum.

The protective layer is deposited over and covers the diaphragm, and protects the diaphragm from damage due to the flow of blood. For example, the layer can prevent the diaphragm from being dissolved by a chemical reaction with the patient's blood. Additionally, the layer impedes biological deposits from forming directly on the diaphragm. In embodiments where the optical sensor assembly is a pressure sensor, the layer transmits pressure from the blood to the diaphragm so that the blood pressure can be sensed. The layer may include a material capable of being deposited onto the diaphragm. For example, the layer may be made from platinum. In some embodiments, the layer may have a thickness of about 0.3 nanometers (nm). In other embodiments, the layer has a thickness of about 0.3 nm or greater. For example, the layer may have a thickness of 0.31 nm, 0.32 nm, 0.33 nm, 0.34 nm, 0.35 nm, >0.35 nm, or any suitable thickness. In certain embodiments, the layer has a thickness of about 0.3 nm or less. For example, the layer may have a thickness of 0.29 nm, 0.28 nm, 0.27 nm, 0.26 nm, 0.25 nm, <0.25 nm, or any suitable thickness. The optical sensor assembly can include any number of additional protective layers deposited over the diaphragm, for example, 1, 2, or 3 protective layers. However, as will be appreciated, any suitable number of protective layers may be used. In some embodiments, all protective layers may be made of the same material. In other embodiments, the protective layers may be made of different materials. As will also be appreciated, in embodiments with at least one protective layer, each of the protective layers may have different thicknesses. As noted herein, platinum silicide is an advantageous protective layer, due to its biocompatibility. For example, platinum silicide has greater resistance to chemical damage from the patient's blood and thus provides greater protection and/or durability for the diaphragm.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

1. An optical sensor assembly for use in a blood pump assembly, the optical sensor assembly comprising:
a visor having an inner surface and an outer surface;
an optical sensor disposed within the visor, the optical sensor comprising an optical sensor inner surface and a diaphragm disposed over a cavity in a substrate, wherein the diaphragm has an exposed surface and exposed sides; and
a protective layer being deposited over and covering the exposed surface and exposed sides of the diaphragm,
wherein the protective layer is one of a platinum-containing layer, a platinum silicide layer, or parylene
and wherein the optical sensor optionally comprises a dot.

2. The optical sensor assembly of claim 1, wherein the protective layer is platinum silicide.

3. The optical sensor assembly of claim 1, wherein the protective layer has a thickness of about 0.3 nanometers (nm) or greater.

4. The optical sensor assembly of claim 3, wherein the thickness of the protective layer is about 0.31 nm to at least about 0.35 nm or greater.

5. The optical sensor assembly of claim 3, wherein the protective layer has a thickness of about 0.3 nm or less.

6. The optical sensor assembly of claim 5, wherein the protective layer has a thickness of about 0.29 nm to about 0.25 nm or less.

7. The optical sensor assembly of claim 1, wherein the protective layer comprises a plurality of layers.

8. The optical sensor assembly of claim 7, wherein the plurality of protective layers are the same protective layer material or different protective layer material.

9. The optical sensor assembly of claim 8, wherein each of the plurality of protective layers has a thickness and that thickness is either the same as or different from the thickness of the other protective layers.

10. A blood pump assembly comprising:
a pump,
a motor,
a rotor,
a pump housing,
a cannula,
an atraumatic extension, and an optical sensor assembly comprising:
a visor having an inner surface and an outer surface;
an optical sensor disposed within the visor, the optical sensor comprising an optical sensor inner surface and a diaphragm disposed over a cavity in a substrate, wherein the diaphragm has an exposed surface and exposed sides; and
a protective layer being deposited over and covering the exposed surface and exposed sides of the diaphragm,
wherein the protective layer is one of a platinum-containing layer, a platinum silicide layer, or parylene
and wherein the optical sensor optionally comprises a dot.

11. The blood pump assembly of claim 10, wherein the optical sensor assembly is affixed to the pump housing.

12. The blood pump assembly of claim 11, wherein the optical sensor assembly is affixed to the pump housing with a glue or epoxy.

13. A method of fabricating an optical sensor comprising:
defining a cavity;
forming a silicon layer over a glass substrate via masking; and
forming a protective layer over the silicon layer and over an uncovered edge portion of the silicon layer.

14. The method of claim 13, wherein the protective layer is one of a platinum-containing layer, a platinum silicide layer, or parylene.

15. The method of claim 14, wherein the protective layer is platinum silicide.
